(19) United States
(12) Patent Application Publication
(10) Pub. No.: US 2012/0220658 A1
(43) Pub. Date: Aug. 30, 2012

(54) Method for Treating Sepsis or Septic Shock

(75) Inventors: Kingsley Yin, Cherry Hill, NJ (US); Ana Rodriguez, Marlton, NJ (US); Bernd W. Spur, Marlton, NJ (US); Jean Walker, Aston, PA (US)

(73) Assignee: University of Medicine and Dentistry of New Jersey, Somerset, NJ (US)

(21) Appl. No.: 13/503,494

(22) PCT Filed: Oct. 21, 2010
(86) PCT No.: PCT/US10/53472

§ 371 Date: May 14, 2012

Related U.S. Application Data
(60) Provisional application No. 61/253,702, filed on Oct. 21, 2009.

(51) Int. Cl. A61K 31/202; A61P 31/00

(52) U.S. Cl. 514/557

(57) ABSTRACT

The present invention is a method for treating sepsis or septic shock using Lipoxin A4.

### INTRODUCTION

This application claims benefit of priority to U.S. Provisional Application Ser. No. 61/253,702, filed Oct. 21, 2009, the content of which is incorporated herein by reference in its entirety.

### BACKGROUND OF THE INVENTION

Sepsis is a major medical problem in the United States. It is estimated that approximately 750,000 new cases of sepsis occur each year with mortality rates approaching 30%. Despite intensive research there are still no adequate treatments for sepsis or septic shock.

It has been postulated that the major mechanism underlying the morbidity and mortality associated with sepsis is a profound activation of the innate immune system in response to bacterial infection. The attendant increased release of inflammatory mediators such as cytokines, arachidonic acid metabolites and free radicals if sustained, can result in tissue injury, multiple organ failure and death. Alternatively, a sustained inflammatory response may lead to a delayed immunosuppression, which is characterized by an inability of populations of lymphocytes and/or monocytes/macrophages to secrete inflammatory cytokines. In this immunocompromised condition, the host is susceptible to opportunistic infections. Thus, an appropriate inflammatory response is essential for pathogen clearance and prevention of tissue injury or immunosuppression. An appropriate inflammatory response can be characterized as one which clears the infectious agent without causing tissue injury and/or an immunosuppressed state. In this regard, the effect of inhibition of pro-inflammatory mediators may lead to the host becoming immunosuppressed. Accordingly, it may be beneficial to stimulate resolution of inflammation rather than suppress or inhibit the inflammatory response. Resolution of inflammation is a programmed phenomenon that involves inhibition of neutrophil sequestration and increased monocyte/macrophage recruitment to the site of injury. The purpose of this switch is to prevent excessive release of neutrophil-derived inflammatory mediators and increase macrophage phagocytosis of apoptotic neutrophils. However, this increased activity of macrophages is of a non-phlogistic nature where the cells do not increase production of inflammatory mediators.

Research has focused on novel compounds that induce inflammation resolution, but are themselves not immunosuppressive. One of these molecules, Lipoxin A4 (LXA4) is an arachidonic acid product of the interaction between platelets and neutrophils or neutrophil conversion of 15S-hydroxy eicosatetraenoic acid (15S-HETE) released from activated monocytes and epithelial cells. The cellular actions of LXA4 include inhibition of chemotaxis, adherence and transmigration of neutrophils, stimulation of macrophage phagocytosis of apoptotic neutrophils, stimulation of chemotaxis of monocytes and reduction in IL-8 gene expression. Similar to many autacoids, LXA4 acts in its local environment and then is metabolically inactivated. In in vivo models, LXA4 has been shown to inhibit neutrophil infiltration but increase monocyte/macrophage recruitment to the site of injury in the zymosan-induced peritonitis model. It has also been reported to attenuate pro-inflammatory gene expression and reduce severity in a dextran sulfate model of colitis. However, in all these reports, the intervention was a pretreatment. In addition, the studies were conducted using stable LXA4 analogs rather than endogenous LXA4. Although, the use of stable analogs circumvents the issue of metabolic inactivation of LXA4, the longer term effect(s) of these stable analogs in a clinically relevant model inflammation has not been established.

### SUMMARY OF THE INVENTION

The present invention is a method for treating sepsis or septic shock by administering to a subject in need thereof an effective amount of a Lipoxin A4. In one embodiment, the Lipoxin A4 is administered intravenously or intraperitoneally. In another embodiment, treatment accelerates inflammation resolution, accelerates bacterial clearance, and increases survival.

### BRIEF DESCRIPTION OF THE DRAWINGS

FIG. 1 shows subgroup analysis of rats that lived longer than 48 hours. This analysis indicated that LXA4 (LX) treatment significantly increased survival compared to vehicle treated CLP rats, n=9 for CLP, n=7 for CLP+LXA4. *P<0.05 by log-rank test of Kaplan-Meier curves.

FIG. 2 shows that LXA4 reduces blood bacterial load 48 hours after CLP compared to vehicle saline-treated rats. *P<0.05 for n=7 for CLP, n=8 for CLP+LXA4 rats.

FIG. 3 shows that the total number of cells in CLP rats was substantially increased compared to sham controls. Administration of LXA4 increased the total number of peritoneal cells. Furthermore, LXA4 increased the number of monocyte/macrophages in the peritoneal cavity of CLP rats without affecting the number of neutrophils. *P<0.05 compared to sham controls, # P<0.05 compared to CLP, for n=4 sham controls, n=7 CLP rats and n=10 for CLP+LXA4 rats.

FIG. 4 shows LXA4 treatment reduced plasma levels of IL-6, 48 hours after CLP as compared to rats given saline vehicle. *P<0.05 compared to sham controls, # P<0.05 compared to CLP+LX, for n=4 sham controls, n=5 CLP rats, n=8 for CLP+LXA4.

FIG. 5 shows that CLP rats had raised plasma MCP-1 levels compared to sham controls. LXA4 treatment reduced plasma levels of MCP-1, 48 hours after CLP as compared to rats given saline vehicle. *P<0.05 compared to sham controls, # P<0.05 compared to CLP+LX, for n=4 sham controls, n=5 CLP rats, n=8 for CLP+LXA4.

FIG. 6 shows plasma IL-10 levels were raised in CLP rats compared to sham controls. LXA4 treatment significantly decreased plasma IL-10 levels. *P<0.05 compared to sham controls # P<0.05 compared to CLP+LX, for n=4 sham controls, n=5 CLP rats, n=8 for CLP+LXA4.

FIG. 7 shows that LXA4 (8 pg/kg) i.v. given 1 hour after CLP increased survival in CLP rats. P<0.05 for n=17, CLP+LX; n=19, CLP, by log rank test of Kaplan-Meier curves.

### DETAILED DESCRIPTION OF THE INVENTION

It has now been shown that a single, low dose of authentic LXA4 in a clinically relevant model of sepsis, increases monocyte/macrophage recruitment to the peritoneal cavity without affecting neutrophil numbers. These changes were associated with decreased blood bacterial load and IL-6 production. Accordingly, the present invention features of use of authentic LXA4 in the treatment of sepsis and septic shock.

As is conventional in the art, sepsis is characterized by dysregulated systemic inflammation with release of a large amount of inflammatory mediators. Symptoms of sepsis include, but not limited to, arterial hypotension, metabolic acidosis, fever, decreased systemic vascular resistance, tachypnea, organ dysfunction, and septicemia (i.e., organisms, their metabolic end-products or toxins). Septic shock refers to acute circulatory failure resulting from septicemia often associated with multiple organ failure and a high mortality rate. Symptoms of sepsis and septic shock can be determined by quantitative analysis (e.g., fever, etc.) or from a blood test (e.g., bacteremia). In this respect, an effective amount of LXA4 is an amount that causes a reduction in one or more symptoms of sepsis or septic shock, i.e., a qualitative or a quantitative reduction in detectable symptoms, including but not limited to a detectable impact on the rate of recovery from disease.

Those of ordinary skill in the art can readily optimize effective dosages and administration regimens using the data presented herein as well as good medical practice and the clinical condition of the individual patient. In some embodiments, a single dose of LXA4 is in the range of 1 μg/kg to 100 μg/kg. In particular embodiments, the dose employed may be dependent on the route of administration. For example, a dose in the range of 1 μg/kg to 20 μg/kg may be appropriate for i.v. administration, whereas a dose of 20 to 100 μg/kg may be appropriate for i.p. administration.

In particular embodiments, LXA4 is of use in the treatment of sepsis or septic shock resulting from septicemia (i.e., organisms, their metabolic end-products or toxins in the blood stream), including bacteremia (i.e., bacteria in the blood). It is further contemplated that the instant method finds application in the treatment of sepsis or septic shock resulting from toxemia (i.e., toxins in the blood), including endotoxemia (i.e., endotoxin in the blood); fungemia (i.e., fungi in the blood); viremia (i.e., viruses or virus particles in the blood); and parasitemia (i.e., helminthic or protozoan parasites in the blood).

According to the method of the present invention, a subject in need of treatment, i.e., a subject exhibiting one or more signs or symptoms of sepsis or septic shock, is administered an effective amount of LXA4 so that the sepsis or septic shock is treated. As the results herein demonstrate, a single, low dose of LXA4 administered from 1 to 5 hours after onset of sepsis accelerates inflammation resolution and bacterial clearance and increases survival. Accordingly, in particular embodiments, the instant method is carried out within the first 1, 5, 12, 24 or 48 hours of onset of sepsis or septic shock, e.g., as determined by the appearance of one or more symptoms of sepsis or septic shock.

Authentic or native LXA4 can be obtained by any conventional method. For example, LXA4 can be derived enzymatically from arachidonic acid, or synthesized from butadiene (Rodríguez, et al. (2000) Tetrahedron Lett. 41:823-826) or d-isoascorbic acid (Gravier-Pelletier, et al. (1991) Tetrahedron Lett. 32:1165-1168). In particular embodiments of the present invention, the LXA4 used in the instant method is authentic or native LXA4 and does not include derivatives or analogs of LXA4. Moreover, for use in the instant invention, the LXA4 is desirably isolated and purified, e.g., to greater than 90%, 95%, 97%, 98% or 99% purity.

For therapeutic applications, particular embodiments embrace the use of LXA4 in a mixture with a pharmaceutically acceptable carrier such as, for example, physiologically compatible buffers such as, but not limited to, physiological saline, a mixture of saline and glucose, heparinized sodium-citrate-citric acid-dextrose solution, alcohols, dimethylsulfoxide (DMSO), and other such acceptable carriers. Sterile injectable solutions can be prepared by incorporating LXA4 in the required amount in the appropriate solvent with various other ingredients, as needed, followed by filtered sterilization. Dispersions also can be prepared in glycerol, liquid polyethylene glycols, and mixtures thereof, and in oils. Generally, dispersions can be prepared by incorporating the various sterilized active ingredients into a sterile vehicle that contains the basic dispersion medium and any required other ingredients conventionally used in pharmaceutical formulations. In the case of sterile powders for the preparation of sterile injectable solutions, methods of preparation include vacuum drying and freeze-drying techniques that yield a powder of the active ingredient plus any additional desired ingredient from a previously sterile-filtered solution thereof. The optimal pharmaceutical formulation will be determined by one of skill in the art depending on the route of administration and the desired dosage. See, for example, Remington: The Science and Practice of Pharmacy, 21st Ed. (2005) Lippincott Williams & Wilki.

LXA4 can be administered via various routes including, but not limited to, intravenous, intradermal, intramusclar, intraperitoneal, intrapulmonary (e.g., term release), oral, sublingual, nasal, anal, vaginal, or transdermal delivery. In particular embodiments, administration is via intravenous or intraperitoneal routes.

Treatment can include a single dose or a plurality of doses over a period of time. The frequency of dosing can be dependent on multiple factors including the pharmacokinetic parameters of LXA4, the route of administration and the condition of the subject. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as pharmacokinetic data observed in animals or human clinical trials.

The invention is described in greater detail by the following non-limiting examples.

### Example 1

### Materials and Methods

Synthesis of LXA4. Authentic or native LXA4 (5S,6R,15S-trihydroxy-7E,9E,11Z,13E-Eicosatetraenoic acid) is known in the art and has the following structure.

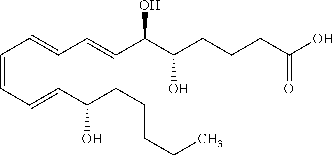

Surgery. Cecal ligation and perforation (CLP) was performed on male Sprague-Dawley rats (250-350 g) according to known methods (Chaudry, et al. (1979) Surgery 85:205-21). The CLP model of sepsis is an established, clinically relevant model of sepsis (Rittirsch et al., (2009) Nature Protocol 4(1):31-36). Animals were fasted overnight before surgery. On the day of surgery, rats were anesthetized with Isofluorane (+oxygen). A 2-cm-long midline incision was made in the abdomen to expose the cecum. Fecal matter was massaged into the cecum. The distal two-thirds of the cecum was ligated with 4.0 surgical silk. Using an 18-gauge needle, the cecum was punctured twice. Some fecal matter was massaged out of the cecum through these holes. The cecum was placed back into the abdomen, which was then closed. Saline (2 ml/100 g; s.c.) was injected to replace any fluids lost during surgery. Five hours after surgery, the rats were given either LXA4 (40 μg/kg rat; i.p.) or saline vehicle. This concentration of LXA4 was derived from published in vivo work in rodents (Bannenberg et al., (2004) Br. J. Pharmacol. 143:43-52). At the end of 48 hours, rats were anesthetized with pentobarbital (50 mg/kg, i.p.). A midline incision was made and the peritoneal cavity was lavaged with 20 ml of PBS containing 0.38% Na citrate (as anticoagulant). Four ml blood was withdrawn from the inferior vena cava into 10 ml syringes containing Na citrate (0.38% final concentration).

Two hundred μl of lavage fluid was taken immediately for measurement of bacterial load. The rest of the fluid was passed through a 250 mm (diameter) nylon mesh to remove debris and then centrifuged at 110×g for 7 minutes at 4° C. The supernatant was removed and stored at −80° C. for further analyses. Forty ml red blood cell lysis buffer was added to the cell pellet for 10 minutes with gentle inversion before being centrifuged again at 110×g (7 minutes; 4° C.). Cells were resuspended in 1 ml PBS.

Bacterial Load. Serial dilutions (1:10) of lavage fluid and blood were made and aseptically spread on tryptic soy agar (Fisher) plates. The plates were incubated overnight at 37° C. in an atmosphere containing 5% CO₂. At the end of the incubation period, colony forming units (CFU) were counted.

Peritoneal Cell Differential. Peritoneal cells were counted using an automated hemocytometer (Beckman Coulter Counter; Z4) and then cytospun onto superfrost ++ slides. In order to perform cell differentials, DIFQUIK staining was performed according to manufacturer's instructions. The cell differential was performed by operators blind to the different treatment groups.

Plasma IL-6, MCP-1 and IL-10 Levels. Plasma IL-6, MCP-1 and IL-10 levels were measured using commercially available kits.

Statistical analysis. Differences between groups for measurements were obtained using Student's t-test, where P<0.05 was taken as significant. If data were non-parametric, a Mann-Whitney U test was used. All data was expressed as mean±S.E.M.

### Example 2

### LXA4 Accelerates Inflammation Resolution and Bacterial Clearance

In this study, the effect of LXA4 in the clinically relevant cecal ligation and puncture (CLP) model of sepsis was analyzed. CLP rats were given either saline or LXA4 (40 μg/kg, i.p.) five hours after surgery.

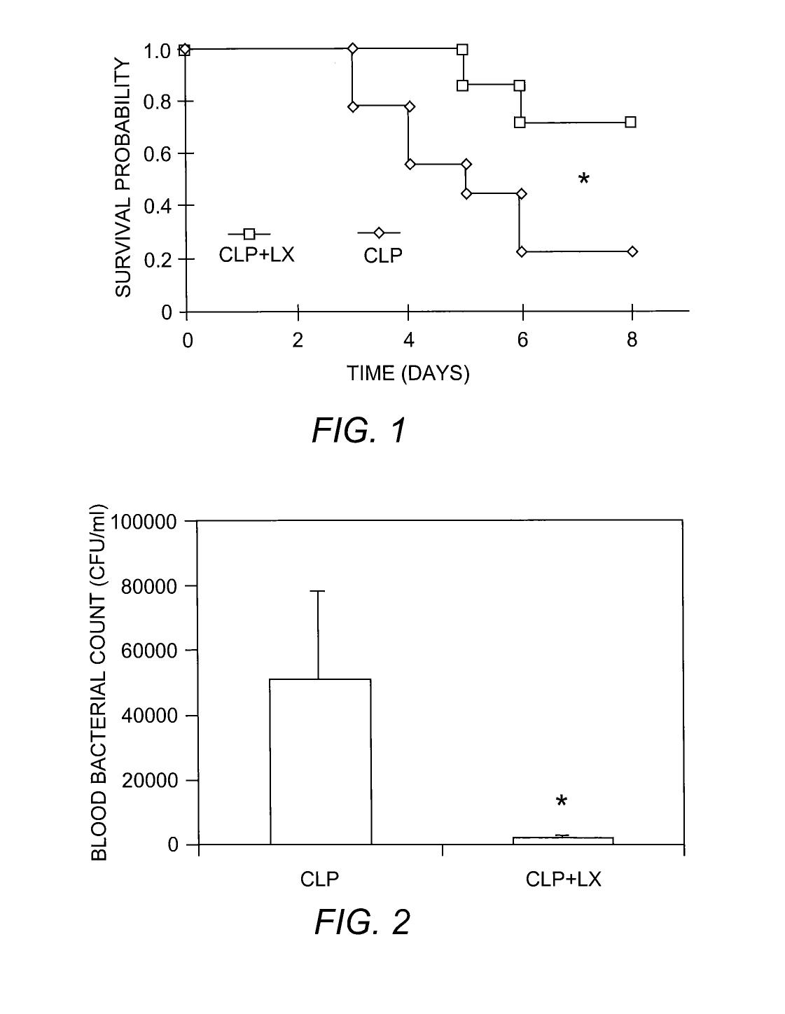

Survival. Kaplan-Meier survival curves were constructed to analyze the effects of LXA4 administration on survival in CLP rats. Subgroup analyses (log rank test) of rats that survived 48 hours, (taken from the same study) revealed that LXA4 administration increased survival rate (FIG. 1). As there was a significant change in the slope of the individual survival curves starting at 48 hours, the effects of LXA4 on systemic inflammatory mediator release, bacterial load and peritoneal leukocyte counts 48 hours after CLP sepsis were analyzed.

Bacterial Load. Bacterial load in blood and peritoneal lavage fluid was measured after plating of serially diluted samples on tryptic soy agar plates. There was clear evidence of bacterial colony forming units (CFU) in blood of CLP rats 48 hours after surgery (FIG. 2). Administration of LXA4 5 hour after surgery substantially reduced blood bacterial load (FIG. 2). There were also high levels of bacteria in the peritoneal cavity 48 hours after CLP surgery. LXA4 administration reduced bacterial load in the peritoneal cavity but the reduction did not reach significance.

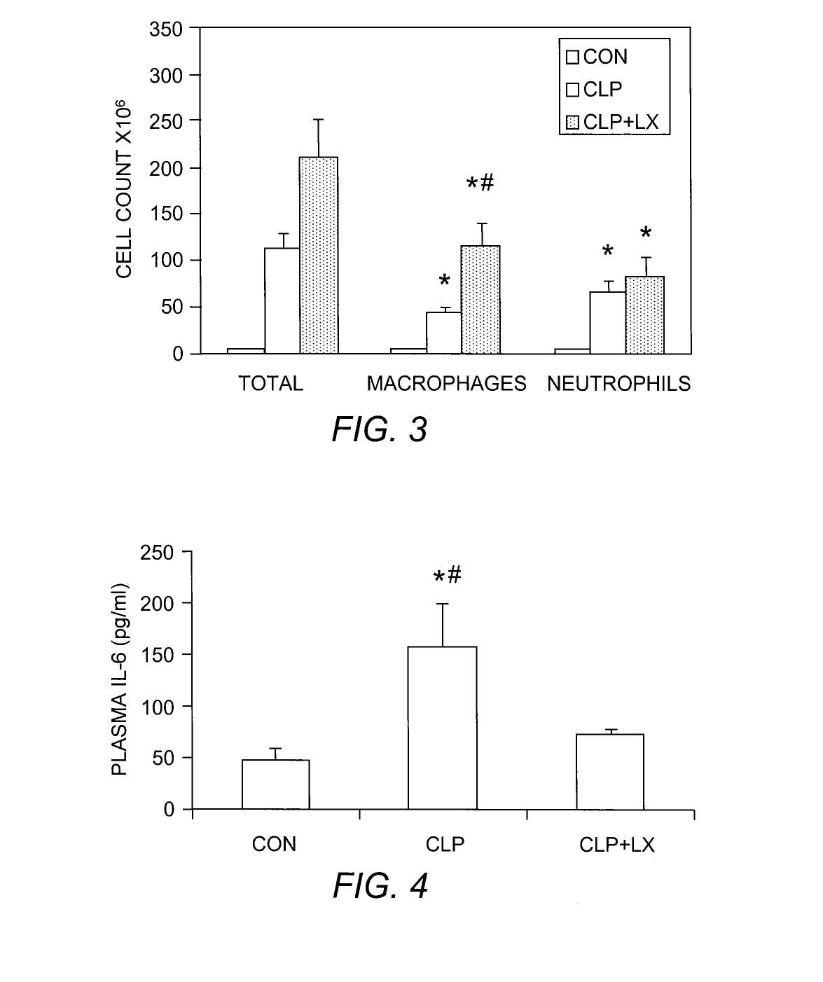

Peritoneal Leukocytes. The total number of peritoneal leukocytes from CLP rats was greater than sham controls. Administration of LXA4 increased peritoneal leukocytes further (FIG. 3). To examine cellular changes in cell infiltration, differential cell counts were obtained after DIFFQUIK staining of cells cytospun onto glass slides. LXA4 administration increased the number of monocytes/macrophages recruited into the peritoneal cavity as compared to CLP rats given saline vehicle, while the number of neutrophils remained similar in both groups.

Plasma IL-6. Plasma IL-6 is postulated be an inflammatory biomarker and is associated with increased mortality in sepsis (Remick, et al. (2005) Infection Immunity 73:2751-2757). Plasma IL-6 levels in CLP rats given vehicle saline were substantially increased compared to sham controls. Administration of LXA4 reduced plasma IL-levels as compared to CLP rats given saline vehicle (FIG. 4).

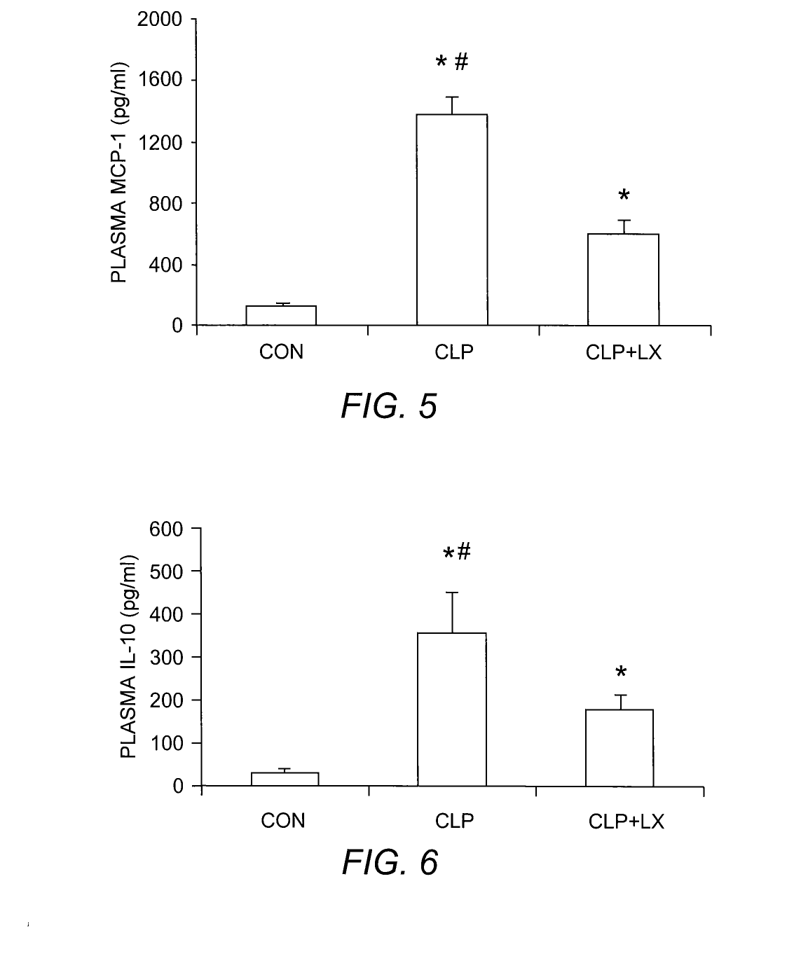

Plasma MCP-1. Plasma MCP-1 has been reported to be an indicator of mortality in severe sepsis as well as delayed mortality in the CLP model of sepsis. Accordingly, plasma MCP-1 was measured as another marker of systemic inflammatory response. Plasma MCP-1 levels were raised in CLP rats compared to sham controls (FIG. 5). LXA4 significantly reduced plasma MCP-1 levels compared to CLP rats given vehicle saline.

Plasma IL-10. Plasma IL-10 is an anti-inflammatory cytokine which is thought to be a mediator of the immunosuppression observed in sepsis. In addition, there is evidence that LXA4 produces some of its anti-inflammatory effects via stimulation of IL-10 production. However, analysis of plasma IL-10 levels in the instant study showed that LXA4 administration decreased plasma IL-10 levels (FIG. 6).

This analysis indicated that LXA4 given five hours after onset of sepsis increased survival of CLP rats which lived past 48 hours. As the LXA4 was given after onset of sepsis, the data clearly showed a clinically relevant effect of LXA4 on survival after sepsis. At this time point, LXA4 decreased both blood bacterial load as well as inflammation (lowered IL-6, MCP-1 and IL-10 levels). Without being bound by theory, the mechanism for this beneficial effect is likely through the proresolution action of LXA4, which increases macrophage recruitment without increased systemic cytokine release. This analysis indicated that LXA4 increased survival and reduced inflammation without compromising the host's ability to clear infection.

### Example 3

### Effects of Intravenous LXA4 Administration on Survival in Sepsis

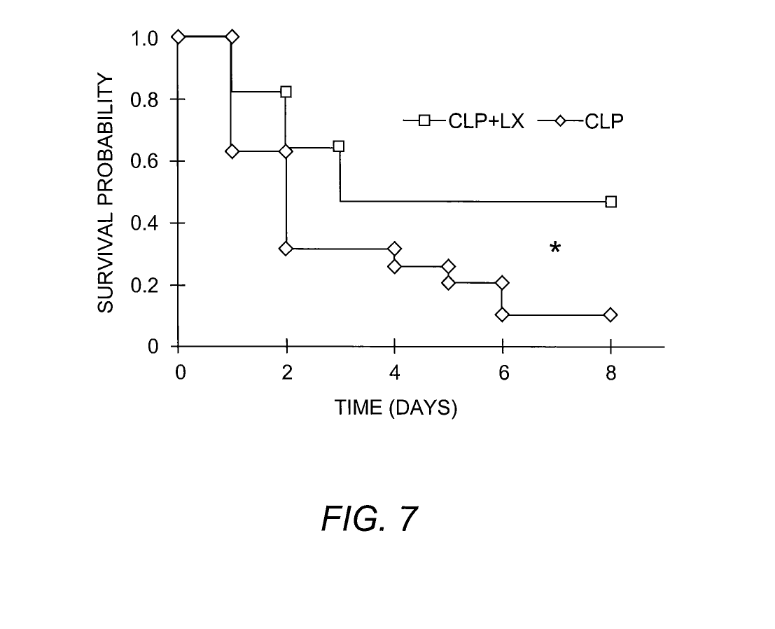

To further examine the use of LXA4 in the treatment of sepsis, the effect intravenous LXA4 administration on survival after CLP-induced sepsis was examined. In these studies, LXA4 (8 μg/kg) was administered intravenously 1 hour after CLP. Survival was monitored every 24 hours for 8 days (FIG. 7). These results showed that a single dose of LXA4 given i.v. 1 hour after CLP-induced sepsis significantly increased survival (P=0.001).

What is claimed is:

1. A method for treating sepsis or septic shock comprising administering to a subject in need thereof an effective amount of Lipoxin A4 thereby treating the subject's sepsis or septic shock.

2. The method of claim 1, wherein the Lipoxin A4 is administered intravenously or intraperitoneally.

3. The method of claim 1, wherein treatment accelerates inflammation resolution, accelerates bacterial clearance, and increases survival.
